(19) United States
(12) Patent Application Publication
(10) Pub. No.: US 2010/0166822 A1
(43) Pub. Date: Jul. 1, 2010

(54) Adhesive cartilage implant

(75) Inventor: Lin Song, Wayne, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Howmedica Osteonics Corp., Mahwah, NJ (US)

(21) Appl. No.: 12/319,129

(22) Filed: Dec. 31, 2008

(51) Int. Cl. A61F 2/00; C12N 5/06; A61K 35/12

(52) U.S. Cl. 424/423; 435/402; 424/93.7

(57) ABSTRACT

A method of producing a cartilage implant which may include coating a substrate with a thermo-sensitive polymer, seeding the polymer with a cell suspension comprising cells that may differentiate into chrondocyte-like cells, wherein the seeding may be conducted at a first temperature, culturing the cell suspension at an increased temperature, whereby cells in the suspension may adhere to the polymer and differentiate into chrondrocyte-like cells, which may form a cartilagenous implant, and decreasing the temperature of the polymer which may allow detachment of the implant from the polymer. The present invention may also include an implant based on this method. The invention may further include a cartilage implant for use in a patient in need thereof, which may include a random three-dimensional configuration of differentiated cells, which may have a natural adhesion surface on at least a portion of the outer surface of the three-dimensional configuration.

### FIELD OF THE INVENTION

The present invention relates to the field of medical technology and is generally directed to a cartilage implant and methods of making cartilage implants.

### BACKGROUND OF THE INVENTION

Cartilage is an avascular connective tissue made up of collagen and/or elastin fibers, and chondrocytes, all of which are embedded in a matrix. There are three main types of cartilage: elastic, fibrocartilage, and hyaline. Elastic cartilage is found in the outer ear and the epiglottis. Fibrocartilage is found between the bones of the spinal column, hips and pelvis. Hyaline cartilage can be found on the ends of bones which form joints, on the ends of the ribs, on the end of the nose, on the stiff rings around the windpipe, and supporting the larynx. Articular cartilage is a specialized type of hyaline cartilage which covers the surface of joints and provides a durable low friction surface that distributes mechanical forces and protects the joint's underlying bone.

Different types of collagen can be found in varying amounts in the collagen matrix, depending on the type of tissue. For example, hyaline cartilage, which is found predominantly in articulating joints, is composed mostly of type II collagen with small amounts of types V, VI, IX, X, and XI collagen also present. On the other hand, fibrocartilage, which can also be found in joints, is primarily composed of type I collagen. Additionally, the fibrocartilaginous tissue that sometimes replaces damaged articular cartilage is composed of type I collagen.

Loss of or damage to cartilage can lead to painful conditions such as osteoarthritis. Damage to cartilage can be caused by traumatic injury, disease and/or age. Since cartilage lacks nerves and blood vessels, it has very limited regenerative capabilities compared to other tissues. Consequently, the healing of damaged joint cartilage results in a fibrocartilaginous repair tissue that lacks the structure and biomechanical properties of normal cartilage. Over time, the repair tissue degrades and leaves damaged joint cartilage, which causes osteoarthritis and reduced movement in the joint.

Various implants exist which serve to repair cartilage injuries and damage. One type of cartilage implant is live cartilage tissue constructed ex vivo, which is then implanted at the site of cartilage damage. Current methods of constructing such an implant involve seeding cells onto a three-dimensional scaffold and allowing them to proliferate into a three-dimensional structure supported by the scaffold.

Another method includes allowing a three-dimensional cluster of cells to float freely in a culture, whereby the cells proliferate to form a three-dimensional implant. The implant is then removed from the culture through chemical or physical means.

Alternatively, the implant may be constructed by seeding cells onto a substrate and allowing the cells to proliferate into a two-dimensional cell sheet. Once the implant is formed, the cell structure is removed from the substrate through chemical or mechanical means, such as enzymatic digestion of adhesive molecules, such as integrins, or removal by physical force.

These methods typically result in the removal of integrins and other adhesion molecules from the surface of the cell structure contacting the substrate. Thus, when these implants are implanted into a cartilage defect, fixation devices such as sutures, screws, chemical adhesives (e.g., fibrin glue) must be used to ensure the implant remains in place. Such fixation devices can cause damage to the surrounding tissue, interfere with the healing process, or can easily fail. For example, fibrin glue has a fast degradation rate, such that the glue can degrade from the site faster than the implant can be secured within the defect site.

Thermo-sensitive polymers have also been used to coat a substrate to which a two-dimensional cell sheet may attach. In order to form a three-dimensional implant, numerous cell sheets must be placed on top of one another, and typically, an adhesive, including the thermo-sensitive polymer itself, may be needed to adhere the cell sheets to one another.

There is a need for methods for repairing cartilage defects which do not require such fixation devices.

### BRIEF SUMMARY OF THE INVENTION

The present invention includes an implant that can be used to repair cartilage and methods of producing the implant. The invention also includes a method of treating cartilage defects using the implant.

In one embodiment, a method of producing a cartilage implant may include coating a substrate with a thermo-sensitive polymer, seeding the polymer with a cell suspension comprising cells that may differentiate into chrondocyte-like cells, wherein the seeding may be conducted at a first temperature, culturing the cell suspension at an increased temperature, whereby cells in the suspension may adhere to the polymer and differentiate into chrondrocyte-like cells, which may form a cartilagenous implant, and decreasing the temperature of the polymer which may allow detachment of the implant from the polymer.

The temperature of the thermo-sensitive polymer during cell seeding may be at a nonphyisiological temperature (e.g., less than about 28° C.), and the cell adhesion, proliferation and/or differentiation may occur upon increasing the temperature to a physiological temperature (e.g., about 36.1° C. to about 37.8° C.). The cells may be, for example, stem cells or human bone marrow stromal cells, or the like. The cells may be placed on the thermo-sensitive polymer in a random three-dimensional configuration. The implant may be detached from the polymer by decreasing the temperature of the polymer back to a nonphysiological temperature such that the implant may detach from the polymer while preserving a natural adhesion surface on at least a portion of the cells which contacted the polymer. The natural adhesion surface may include integrins. The implant, with its natural adhesion surface, may be transferred from the substrate and polymer to a patient in need thereof.

In a further embodiment, the present invention may also include an implant based on this method.

In yet another embodiment, a cartilage implant for use in a patient in need thereof may include a random three-dimensional configuration of differentiated cells, which may have a natural adhesion surface on at least a portion of the outer surface of the three-dimensional configuration. In this embodiment, the natural adhesion surface may include integrins.

### BRIEF DESCRIPTION OF THE DRAWINGS

FIG. 1 illustrates one embodiment of a three-dimensional configuration, or construct, of cells, embedded in agarose.

FIG. 2 illustrates a cross-section view of one embodiment of a three-dimensional cell configuration, having undergone H&E staining, which shows the three-dimensional shape of the cell configuration.

FIG. 3 illustrates a cross-section view of yet another embodiment of a three-dimensional cell configuration, stained with Safranin O, wherein the cells are embedded in a sulfated proteoglycan rich ECM, and show characteristics of cartilage-like tissue.

FIG. 4 illustrates a cross-section view of another embodiment of a three-dimensional cell configuration, stained with Alcian blue, wherein the cells are embedded in a sulfated proteoglycan rich ECM, and show characteristics of cartilage-like tissue.

FIG. 5 illustrates a cross-section view of yet a further embodiment of a three-dimensional cell configuration, stained positive with integrin β1 antibody, which shows expression of adhesion molecules, particularly on the side of the configuration (e.g., the bottom area) closest to the thermo-sensitive polymer coating.

FIG. 6 illustrates a cross-section view of one embodiment of a three-dimensional cell configuration, stained positive with integrin α5 antibody, which shows expression of adhesion molecules, particularly on the side of the configuration (e.g., the bottom area) closest to the thermo-sensitive polymer coating.

### DETAILED DESCRIPTION

The present invention is directed to a cartilage implant which may include natural surface adhesion molecules. The present invention is also directed to a process and method of making such a cartilage implant.

In one embodiment, a method for making a cartilage implant may include a substrate on which a thermo-sensitive material is coated. The substrate may be any structure or material known in the art capable of forming a solid surface on which cells may adhere.

The thermo-sensitive material may be, for example, any polymer having the physical property of becoming alternatively adherent and nonadherent. Specifically, the thermo-sensitive polymer may be selectively adherent and nonadherent to cells placed in contact with it depending upon the temperature to which the polymer is exposed. Thus, adjusting the polymer to a first temperature may allow cells to adhere to the polymer, while adjusting the polymer to a second temperature may not allow cells to adhere to the polymer.

In a preferred embodiment, the temperature at which cells can adhere to the polymer may be equivalent to a “physiological temperature” at which cells may proliferate. Alternatively, the temperature at which cells can no longer adhere to the polymer may be equivalent to a “nonphysiological temperature” at which cell proliferation is not possible. For example, the physiological temperature at which the polymer may adhere to cells may be about 36.1° C. to about 37.8° C., and preferably about 37° C., and the nonphysiological temperature at which the polymer may not adhere to cells may be below this range, and more specifically less than about 28° C., and in one embodiment, it may be about 20° C., and in another embodiment, it may be about 4° C. These temperatures may be varied within the physiological range of a specific type of cell, and may further be adjusted to accommodate different types of thermo-sensitive polymers which may alternately adhere and not adhere at temperatures other than about 36.1° C. to about 37.8° C. and less than about 28° C., respectively.

The thermo-sensitive polymer may be any polymer or copolymer, synthetic or natural or a combination of both, which has the physical property of being selectively adherent or nonadherent to cells, as well as being biocompatible, non-cytotoxic, biologically inert, and be capable of supporting proliferation, differentiation, and metabolic activity. Such polymers may be chosen among, for example, poly(N-isopropylacrylamide), polycaprolactone, 2-ethoxyethyl ether and 2-phenoxyethyl vinyl ether copolymer, or the like. These, as well as other similar polymers and copolymers, may, at a first temperature, adhere to cells, while as a second temperature, may not adhere to cells. This physical property may be regulated and adjusted by altering the temperature of the thermo-sensitive polymer.

The temperature of the thermo-sensitive polymer may be adjusted through any means required, such as adjusting the temperature of the substrate directly, adding a liquid such as deionized water of a specified temperature directly onto the polymer where the cells are connected, adjusting the temperature of the culture medium, or by any other method known to those skilled in the art.

In one embodiment, a cell suspension may be seeded onto the thermo-sensitive polymer, such that the cells within the cell suspension interact with one another to form a three-dimensional configuration. The cell suspension may be placed on a substrate, for example a culture vessel or culture disk, having a thermo-sensitive polymer coating along the culture surface, or the interior of the vessel where culturing will take place. The cell suspension may have a high density of cells, such that the cells interact with one another to form a three-dimensional configuration in which at least a portion of the cells are in immediate contact with the thermo-sensitive polymer.

The three-dimensional configuration of the cells may be random, in that the cells interact with one another, upon addition into the culture vessel, such that they randomly disperse above the thermo-sensitive polymer coating. At least a portion of the cells may be in immediate contact with the polymer, while the remainder of the cells may be randomly positioned above those cells in immediate contact with the polymer. Thus, the cells may form a random, three-dimensional configuration, which may closely resemble natural cartilage tissue following differentiation and proliferation.

In one embodiment, the thermo-sensitive polymer may modify cell attachment as to the portion of the cells in immediate contact with the polymer. For example, if the temperature of the polymer is not at a physiological temperature (e.g., less than about 28° C.), the cells will not adhere to the polymer and will also not differentiate or proliferate. In another example, if the temperature of the polymer is at a physiological temperature (e.g., about 36.1° C. to about 37.8° C.), at least the portion of the cells in immediate contact with the polymer may begin to express adhesion molecules, such as integrins, and may attach to the polymer coating, thereby anchoring the three-dimensional configuration of cells within the culture vessel.

The cells used in this invention may be any type desired, including, but not limited to, stem cells, progenitor cells, embryonic stem cells, bone marrow cells, mesenchymal cells, chondrocytes, chondroblasts, osteoblasts, or combinations of these cells. In a preferred embodiment, the cells may be human bone marrow stromal cells. In one embodiment, the cells may already be formed into a three-dimensional configuration, which may then be transferred to the substrate having the thermo-sensitive polymer coating to have the cells begin to produce adhesion molecules, such as integrins.

Any cells used may be retrieved from various sources, including the patient to be treated, other patients of the same species, pools of cells from other patients or animals, individual animals, or commercially available cell lines. Cells may be unaltered and seeded immediately after removal from the source or remain in culture until utilized. The cells may be allogenic, autogenic, or xenogenic to the patient to be treated. Combinations of cells may be used.

The cells may be placed within the culture vessel with a culture medium. For example, bone marrow stromal cells may be added to the culture vessel with an initial medium which may contain components of extracellular matrix (ECM), such as fibronectin and laminin, which may assist in cell adhesion. Once the cells are securely attached in this medium, the medium may be switched to one that induces cartilage formation. Such culture mediums are readily known in the art. Of course, if this embodiment is undertaken using, for example, chondrocytes, a medium containing components of extracellular matrix may not be required since the cells can adhere satisfactorily without the presence of ECM.

If the culture vessel, in particular the thermo-sensitive polymer, is at a physiological temperature, such as about 36.1° C. to about 37.8° C., at least a portion of the cells, assisted by the culture medium, may adhere to the polymer and may proliferate and differentiate.

In one embodiment, the thermo-sensitive polymer may already be at a physiological temperature, such that once the cells are seeded, they may immediately adhere to the polymer and may begin differentiation and proliferation.

In another embodiment, the thermo-sensitive polymer may be at a nonphysiological temperature. In this embodiment, the cells are seeded onto the substrate and polymer, but they may not adhere, differentiate or proliferate. Once the cells are seeded, the temperature of the polymer may be increased to a physiological temperature, whereby the cells may then adhere to the polymer and may proliferate and differentiate.

In a further embodiment, once the three-dimensional configuration of cells has differentiated into chondrocyte-like cells and proliferated to a sufficient degree to constitute a three-dimensional tissue-like implant of the required size, the temperature of the thermo-sensitive polymer may be reduced to the nonphysiological temperature (e.g., less than about 28° C.) for the specific cells and polymer used. The temperature of the polymer may be reduced using any of the methods described above.

At the nonphysiological temperature, differentiation and proliferation processes are halted, and the polymer may become nonadherent as to the cells. Thus, the three-dimensional configuration of cells, now a three-dimensional implant, or tissue construct, may be released from the thermo-sensitive polymer coating on the substrate (e.g., culture vessel). Such detachment of the three-dimensional implant does not require use of chemical processes (e.g., enzymatic degradation) or physical processes (e.g., the use of an instrument to physically pull the cells from the substrate), which are typically used in the cell culture art.

Since the cells detach from the substrate merely by a change in temperature, the adhesion molecules, for example integrins, may remain on the surface of the three-dimensional implant that was in immediate contact with the thermo-sensitive polymer. The adhesion molecules may remain, thus creating a natural adhesion surface which may serve as a biological adhesive when the three-dimensional implant is introduced into a defect site, preferably a chondral defect, in a patient in need thereof.

In the prior art, discussed above, where chemical and physical means are used to remove a cell/tissue structure from a substrate, adhesion molecules are lost. As such, secondary adhesives such as, for example, sutures, screws, tabs, staples, or chemical adhesives such as fibrin glue are required in order to secure the implant within a defect site. These secondary adhesives can cause problems within a defect site, such as interfering with the healing process or causing infection or irritation. Additionally, chemical adhesives such as fibrin glue may have a fast degradation rate, and thus only provide for temporary fixation of the implant. Such secondary adhesives are not required with the present invention as the natural adhesion surface obtained through the above method may be sufficient to secure the three-dimensional implant within a defect site. Of course, secondary adhesives may be utilized where additional anchoring of the implant is required.

In another embodiment, the present invention may include a method of constructing a three-dimensional tissue implant using a substrate, such as a culture vessel or disk, having a coating of thermo-sensitive polymer. A cell suspension containing a high concentration of cells, such as human bone marrow stromal cells may be added to the culture vessel such that at least a portion of the cells is in immediate contact with the thermo-sensitive polymer. The remainder of the cells in the suspension may form a three-dimensional configuration, preferably a random configuration, above the portion of cells in immediate contact with the polymer. The suspension may also include a culture medium which may induce adhesion and/or differentiation and proliferation.

The temperature of the thermo-sensitive polymer, and thus the culture vessel and cell suspension, may then be increased to a physiological temperature, for example about 36.1° C. to about 37.8° C., if the temperature is not already at such a physiological temperature already, at which point the portion of cells in immediate contact with the polymer may express adhesion molecules such as integrins and adhere to the polymer. Additionally, at this temperature, the cells within the three-dimensional configuration may begin differentiation and proliferation. Evaporation, or other similar process, of the culture medium may also occur, at this temperature, such that the concentration of cells in the suspension increases, and the volume of the medium condenses, thereby ensuring the cells closely interact within the three-dimensional configuration.

In a preferred embodiment, the cells may then be allowed to differentiate into chondrocyte-like cells and form extracellular matrix-like material between the cells. Once the desired differentiation, proliferation, and growth of extracellular matrix-like material is obtained, the temperature of the thermo-sensitive polymer, and thus the culture vessel and three-dimensional configuration of cells, may be reduced to a nonphysiological temperature, for example less than about 28° C. At this temperature, differentiation and proliferation may cease to occur, and the polymer may no longer be adherent to the portion of cells in immediate contact with the polymer. The three-dimensional configuration of cells, now a three-dimensional tissue implant, which is no longer adhered to the polymer, may be removed from the culture vessel. The implant may still have a natural adhesion surface comprising adhesion molecules, such as integrins, which may assist in securing the implant within a defect site in a patient in need thereof. The implant, once removed from the culture vessel, may be implanted into the defect site.

In a further embodiment, a live three-dimensional tissue implant may include a three-dimensional configuration of cells oriented in a random configuration, and a natural adhesion surface made of naturally expressed adhesion molecules, such as integrins, from at least a portion of cells within the implant. As discussed in the above methods, the natural adhesion surface forms on at least a portion of cells which were in immediate contact with the thermo-sensitive polymer coating on the substrate on which the implant was grown.

Upon implantation of the three-dimensional implant of the present invention, the adhesion molecules of the natural adhesion surface of the implant may bind to the extracellular matrix within the chondral defect to form a secure, long-lasting, natural anchor for the implant. In one embodiment, the natural adhesion surface is on the bottom surface of the implant, such that the natural adhesion surface may anchor to the extracellular matrix located on the bottom portion of the chondral defect.

Additionally, in one embodiment, the defect site may be prepared for implantation of the implant, whereby the surgeon may debride the chondral defect and remove any calcified cartilage. Subchondral bone is not required to be removed, which provides for even greater chances of success because the risk of implant failure may be reduced since less bone resorption and degradation may occur, and a faster recovery rate may be expected. Of course, the implant may also repair subchondral bone defects in addition to chondral defects, if required.

Once the defect site is repaired, the implant may be press-fit, or may be implanted through other known methods, such that a tight fit between the implant and surrounding extracellular matrix is achieved. The natural adhesion surface of the implant, which may be located on the bottom surface of the implant, may adhere to the collagen and other extracellular matrix components in the surrounding cartilage, or subchondral bone plate, to secure the implant within the defect site.

In a further embodiment, multiple implants may be implanted into a defect site. Multiple implants may be used where a defect is exceptionally large as to width and/or depth. For example, multiple implants may be placed in the defect site on top of one another, where the defect is deep, or may be placed side by side, where the defect is wide, or any combination suitable for a specific defect site shape.

One or more biological agents may be added to the three-dimensional implant, a piece of the implant, a portion of a piece of the implant or a portion of the implant. Likewise, different biological agents may be placed in various portions of the implant or may be placed simultaneously in various portions of the implant. By “biological agent” it is meant any agent that has, or produces, biological, physiological and/or pharmaceutical activity upon administration to a living organism. These biological agents may be added to the implant at any time, for example, before, during or after implantation.

The implant can have varying degrees of biological agent content. The presence of biological agents can be controlled such that growth factor content is maximal or negligible. Biological agent content may vary with depth or location.

Suitable biological agents include, but are not limited to, growth factors, cytokines, antibiotics, antimicrobials, biomolecules, drugs, strontium salts, fluoride salts, calcium salts, sodium salts, bone morphogenetic factors, chemotherapeutic agents, angiogenic factors, anti-inflammatory compounds, such as for example IL-1Ra or TNF-alpha, osteoconductive agents, chondroconductive agents, inductive agents, bisphosphonates, painkillers, proteins, peptides, or combinations thereof. Other biological agents may include cells such as for example allogenic cells, autologous cells, progenitor cells, stem cells, bone marrow stromal cells, mesenchymal cells, fibroblasts, chondrocytes, tenocytes, synovicytes, or the like. Further biological agents may include platelet-rich-plasma (PRP), platelet concentrate, bone marrow concentrate, plasma concentrate, blood, bone marrow, synovial fluid, hyaluronan and hyaluronic acid.

Growth factors that can be added to the implant include platelet derived growth factor (PDGF), transforming growth factor beta (TGFβ), insulin-related growth factor-I (IGF-I), insulin-related growth factor II(IGF-II), beta-2-microglobulin, bone morphogenetic protein (BMP), fibroblast growth factor (FGF), hepatocyte growth factor (HGF), cartilage derived morphogenetic protein (CD-MP), growth differentiation factors (GDFs), or combinations of growth factors.

Chondroinductive agents include prostaglandin E2, thyroid hormone, dihydroxy vitamin D, ascorbic acid, dexamethasone, staurosporine, dibutyrl cAMP, concavalin A, vanadate, FK506, or combinations of different chondroinductive agents. Antibiotics include tetracycline hydrochloride, vancomycin, cephalosporins, and aminoglycocides such as tobramycin, gentamicin, and combinations thereof. Pain killers include lidocaine hydrochloride, bipivacaine hydrochloride, ketorolac tromethamine and other non-steroidal anti-inflammatory drugs.

The biological agent added to the implant may also be a protein or combinations of proteins. For example, proteins of demineralized bone, bone protein (BP), bone morphogenetic protein (BMP), such as BMP5 or the like, osteonectin, osteocalcin, osteogenin, or combinations of these proteins can be added to the implant.

Other suitable biological agents include cis-platinum, ifosfamide, methotrexate, doxorubicin hydrochloride, or combinations thereof.

The methods and implants of the invention can be used to treat any cartilage defect, whether it is in elastic cartilage, fibrocartilage, or hyaline cartilage. For example, the method could be used for cartilage repair in joints, such as a knee, ankle, hip, shoulder, elbow, temporomandibular, sternoclavicular, zygapophyseal, and wrist; or any other place where cartilage is found, such as the ear, nose, ribs, spinal column, pelvis, epiglottis, larynx, and windpipe. The implant may also be used in rhinoplasty procedures, including but not limited to reconstruction via a dorsal septal graft. The implant may be used to repair cartilage during a microtia-atresia surgical correction or in other types of auricular reconstructive procedures, such as those secondary to trauma or cancer.

The implant of the invention can be used to repair cartilage in any patient in need thereof. By “patient” is meant any organism which has cartilage, including, but not limited to humans, monkeys, horses, goats, dogs, cats, and rodents.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

### EXAMPLE 1

In this example, an in vitro study was performed to determine the appropriate cell seeding density in thermo-sensitive polymer coated 96-well culture plates.

Human mesenchymal stem cells (hMSCs) were expanded in DMEM/10% FBS/1% PS at 37° C. (a physiological temperature) up to passage 3. Cells were trypsinized, washed, and resuspended in DMEM/10% FBS/1% PS. Cells were then seeded in the 96-well culture plate coated with thermo-sensitive polymer at the following densities: 1×10⁴ cells/well, 2×10⁴ cells/well, 5×10⁴ cells/well, 1×10⁵ cells/well, and 2×10⁵ cells/well. Following a 24-hour culture at 37° C., the medium was changed to serum-free DMEM supplemented with TGF-β3. Macroscopic examination of cell morphology was conducted daily for 14 days. On the termination day, the culture plate was transferred to 4° C. (a nonphysiological temperature) to release the cell construct from the well.

The results of the study illustrated that within 24 hours of plating, cells became attached to the bottom of the plate, forming a three-dimensional cell configuration or construct. After 14 days, the cell constructs in the wells with initial density of 5×10⁴/well and lower remained attached as a three-dimensional sheet. On the other hand, cell constructs in the wells with initial density of 1-2×10⁵/well contracted and formed a 3-D sphere.

As a result of the study, it was determined that the optimal cell seeding density to form a three-dimensional cell construct is 5×10⁴/well in 96-well plates.

### EXAMPLE 2

In this example, the three-dimensional cell constructs formed in thermo-sensitive polymer coated 96-well culture plates were assessed.

As discussed in Example 1, hMSCs were seeded to form a three-dimensional configuration or construct in a 96-well plate coated with thermo-sensitive polymer at the density of 2×10⁴ cells per well and induced to form cartilage in serum-free DMEM medium supplemented with TGF-β3 for 21 days. Cell constructs were released from the culture plate by incubating at 4° C. for a few minutes. Then the cell constructs were embedded in 1% agarose and subjected to histological processing. Tissue sections of 5 μm were prepared for the following stains: H&E, Safranin O, Alcian Blue (pH 1.0), and integrin α5β1 antibodies.

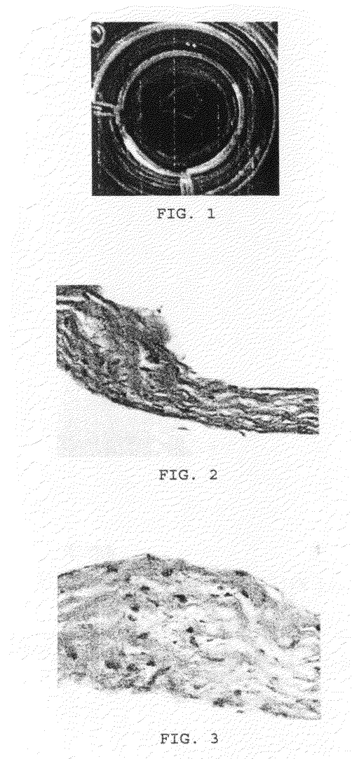

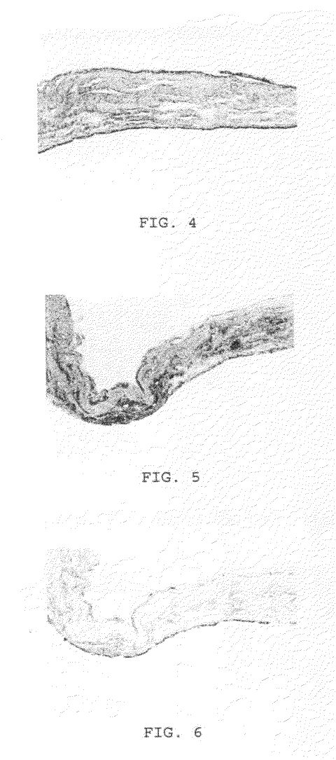

The cell construct remained attached to the plate over 21 days of culture. Once released from the plate, the construct maintained its circular shape (FIG. 1). Upon study of the cross sections, the construct appeared to consist of multiple layered cells (FIG. 2) embedded in a sulfated proteoglycan rich ECM (FIGS. 3 and 4), characteristic of cartilage-like tissue. The constructs were also stained positive with integrin α5β1 (FIGS. 5 and 6), particularly on the side in close contact with the thermo-sensitive polymer coating.

As a result of the study, three-dimensional cell constructs were formed on the thermo-sensitive polymer coated plate. The construct resembled the cartilage like tissue and expressed cell adhesion molecules of integrin α5β1.

1. A method of producing a cartilage implant, comprising:
a) coating a substrate with a thermo-sensitive polymer;
b) seeding the polymer with a cell suspension comprising cells that differentiate into chrondocyte-like cells, wherein said seeding is conducted at a first temperature;
c) culturing the cell suspension at an increased temperature, whereby cells in the suspension adhere to the polymer and differentiate into chrondrocyte-like cells, thus forming a cartilagenous implant; and
d) decreasing the temperature of c), thus allowing detachment of the implant from the polymer.

2. The method of claim 1, wherein the thermo-sensitive polymer is selected from the group consisting of poly(N-isopropylacrylamide), polycaprolactone, 2-ethoxyethyl ether, and 2-phenoxyethyl vinyl ether copolymer.

3. The method of claim 1, wherein the cells are selected from the group consisting of stem cells, progenitor cells, embryonic stem cells, bone marrow cells, human bone marrow stromal cells, mesenchymal cells, chondrocytes, chondroblasts, and osteoblasts.

4. The method of claim 1, wherein the cells are in a three-dimensional configuration.

5. The method of claim 4, wherein the three-dimensional configuration of the cells is random.

6. The method of claim 1, wherein the first temperature is a nonphysiological temperature.

7. The method of claim 6, wherein the nonphysiological temperature is less than about 28° C.

8. The method of claim 1, wherein the increased temperature is a physiological temperature.

9. The method of claim 8, wherein the physiological temperature is about 36.1° C. to about 37.8° C.

10. The method of claim 1, wherein the temperature at which the implant detaches from the thermo-sensitive polymer is a nonphysiological temperature.

11. The method of claim 10, wherein the nonphysiological temperature is less than about 28° C.

12. The method of claim 1, wherein the implant released from the thermo-sensitive polymer comprises an adhesive surface.

13. The method of claim 12, wherein the adhesive surface comprises a natural adhesion surface.

14. The method of claim 13, wherein the natural adhesion surface comprises integrins.

15. The method of claim 1, further comprising e) removing the detached implant from the substrate and thermo-sensitive polymer and implanting the detached implant into a patient in need thereof.

16. A cartilage implant for regenerating joint defects in a patient in need thereof, comprising a three-dimensional configuration of cells produced using the method of claim 1.

17. A cartilage implant for use in a patient in need thereof, comprising a random three-dimensional configuration of differentiated cells, wherein at least a portion of the outer surface of the three-dimensional configuration comprises a natural adhesion surface.

18. The cartilage implant of claim 17, wherein the natural adhesion surface comprises integrins.

19. The cartilage implant of claim 18, wherein the natural adhesion surface further comprises extracellular matrix molecules.

20. The cartilage implant of claim 17, wherein the natural adhesion surface of the implant is capable of adhering to a cartilage defect in a patient in need thereof.
